(19) United States
(12) Patent Application Publication
(10) Pub. No.: US 2011/0060371 A1
(43) Pub. Date: Mar. 10, 2011

(54) SURGICAL APPARATUS FOR OSTEOSYNTHESIS

(76) Inventor: Alexandre Worcel, Montlignon (FR)

(21) Appl. No.: 12/853,597

(22) Filed: Aug. 10, 2010

Related U.S. Application Data
(63) Continuation-in-part of application No. 12/440,382, filed on Jul. 27, 2009.

(30) Foreign Application Priority Data
Sep. 8, 2006 (FR) 0607873

(51) Int. Cl. A61B 17/80

(52) U.S. Cl. 606/286

(57) ABSTRACT

A surgical apparatus for osteosynthesis, includes a plate (2) provided with at least two holes and intended to join two bone parts, and a blocking sleeve (10) which is substantially of revolution and is intended to be screwed into one of the holes of the plate, the blocking sleeve having an axial orifice for passage of a pin to be screwed into one of the bone parts, wherein the lower part of the blocking sleeve has a slit along at least one of its radii, and the blocking sleeve and the associated hole cooperate by threaded engagement in order to block the sleeve in the hole when the sleeve is screwed into the hole.

### RELATED APPLICATIONS

This application is a continuation-in-part of U.S. application Ser. No. 12/440,382, which has a 371 (c) date of Jul. 27, 2009 which is a U.S. National stage entry of International Application No. PCT/FR07/01450, filed Sep. 7, 2007, claims priority to French Application No. 0607873, filed Sep. 8, 2006. The entire contents of each of the foregoing applications are incorporated herein by reference in their entirety.

### FIELD OF THE INVENTION

The present invention relates to a surgical apparatus for osteosynthesis, comprising a plate and a blocking sleeve. It also relates to an apparatus further comprising a pin, the blocking sleeve being intended to ensure blocking between the plate and the pin.

### BACKGROUND OF THE INVENTION

Various devices from the prior art are known which are able to provide an attachment between a pin and a plate.

Thus, the use of conventional type screws has been proposed, the head of which is milled at 45° so that it may be embedded inside a recess of the same shape provided in the plate. However, it is found that the axis of the threaded hole made in the bone portion does not strictly coincide with that of the frustro-conical cavity made in the plate, stresses are generated at the bone portion, the latter being capable over time of undermining the integrity of the bone portion and consequently being detrimental to proper attachment of the plate.

An angular shift of the axes of the pin and of the sleeve was proposed in order to find a remedy to these drawbacks. Such a device is disclosed in document EP-1 583 478 and is illustrated in FIG. 1. This osteosynthesis device comprises a plate 2 including at least two holes, only one of which is illustrated in the figure, intended to receive pins which are screwed into a bone portion.

The holes of the plate area are threaded and receive an intermediate sleeve 6. The latter has a central orifice which is itself threaded, the zz′ axis of which is tilted by an angle α relatively to its axis of revolution yy′.

Finally, a blocking sleeve 10 is screwed by threaded engagement to its lower portion in the intermediate sleeve.

In order to place the osteosynthesis device, the practitioner after having perforated the plate 2 in at least two locations selected for providing its attachment on the bone portion 4, performs drilling of this bone portion by guiding a drill perpendicularly to the plate. Next, he/she places the intermediate sleeve 6 and screws the threaded pin 8 into the bone portion up to the desired length. He/she then introduces the blocking sleeve 10 on the pin 8 and proceeds with screwing this blocking sleeve 10 into the intermediate sleeve 6.

It is understood that because of the angular shift a existing between the respective axes yy′ and zz′, gradual jamming of the blocking sleeve 10 is achieved during this screwing in the intermediate sleeve 6. Irreversible blocking of the pin 8 is thereby achieved relatively to the plate 2.

This device is efficient but it has a certain number of drawbacks.

First of all, tolerance is low on the angular gap between the axes yy′ and zz′. Manufacturing the intermediate sleeve is therefore delicate, which results in high manufacturing cost.

Moreover, this angular gap while ensuring efficient blocking has the drawback that this blocking already occurs right at the beginning of the screwing of the blocking sleeve 10. Therefore, only insufficient penetration of the blocking sleeve into the plate 2 may occur.

Finally, in the case of a plate with a small thickness (for example less than 3 mm), the intermediate sleeve and the blocking sleeve have to be partly inserted into the bone portion so as to allow the device to be properly locked. With this, it is not possible to use this prior art device for the upper bones (arms, forearms, hands) which are too thin.

### SUMMARY OF THE INVENTION

The object of the invention is to do away with the limitations of the osteosynthesis devices of the prior art.

For this purpose, the object of the invention is a surgical apparatus for osteosynthesis comprising a plate provided with at least two holes intended to join two bone portions and with at least one blocking substantially axisymmetrical sleeve, intended to be screwed into one of the holes of the plate, said blocking sleeve including an axial orifice intended for letting through a pin to be screwed into one of the bone portions, wherein: a lower portion of the blocking sleeve includes a slit along at least one of its radii, and said blocking sleeve and the associated hole cooperate by threaded engagement in order to block the blocking sleeve in the hole when the sleeve is screwed into the hole.

Advantageously, the slit of said blocking sleeve is diametrical.

According to a preferred embodiment, said blocking sleeve has in a lower portion a smaller casing than in an upper portion. In particular, said blocking sleeve has a conical or a semi-hemispherical casing.

Alternatively, according to another preferred embodiment, said blocking sleeve has a cylindrical casing and the hole, a beveled casing.

Advantageously, said blocking sleeve and the edges of the holes of the plate have different hardness. In particular, one of the parts among said blocking sleeve and an edge of the plate intended to receive the sleeve is treated in order to increase its hardness, notably by treating its surface with carbon or by work hardening.

According to a particular embodiment, an intermediate sleeve is screwed into the hole of the plate and is intended to cooperate by threaded engagement with the blocking sleeve.

Advantageously, the apparatus according to the invention further includes a pin intended to be screwed into a bone portion. Preferably, the pin is headless.

Preferably, the inner casing of the blocking sleeve and the outer casing of the pin are selected so that the sleeve initially engages with the pin without any friction.

### BRIEF DESCRIPTION OF THE DRAWINGS

The invention will be better understood upon reading the following description of embodiments of the invention, described as non-limiting examples, with reference to the appended drawings, wherein:

FIG. 1, already described, illustrates a surgical apparatus for osteosynthesis according to the prior art;

FIG. 2 illustrates a surgical apparatus for osteosynthesis according to the invention;

FIGS. 3 and 4 respectively illustrate a side view and a bottom view of the blocking sleeve of the apparatus of FIG. 2;

FIG. 5 illustrates an alternative embodiment of the blocking sleeve according to the invention; and

FIG. 6 illustrates another embodiment of a surgical apparatus for osteosynthesis according to the invention.

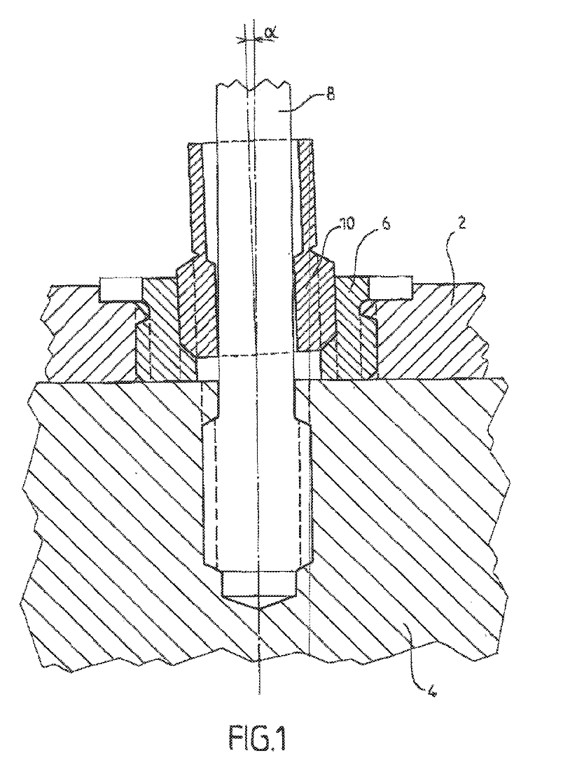

### DETAILED DESCRIPTION OF THE INVENTION

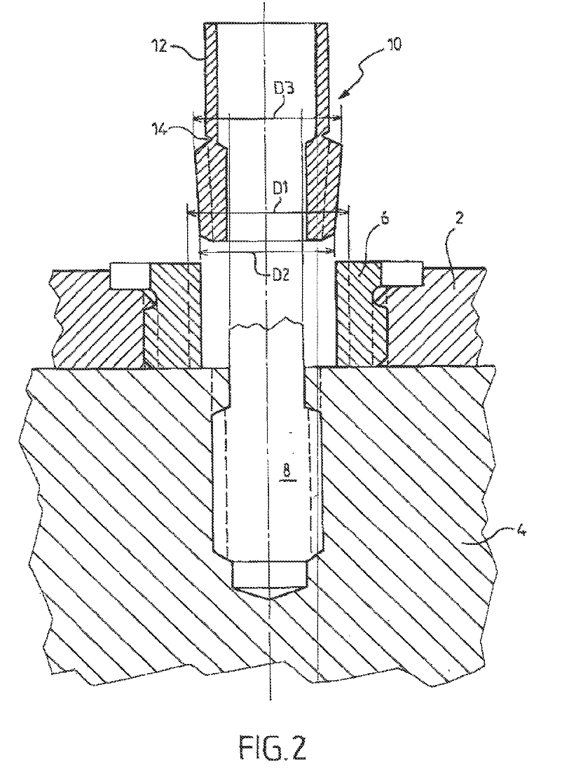

FIG. 2 illustrates a sectional view of a surgical apparatus for osteosynthesis according to the invention. It comprises a plate 2 provided with two holes (only one of which is illustrated) each intended to receive a pin 8 to be screwed into the bone portion 4.

In each hole, the practitioner will introduce a blocking sleeve 10 in order to maintain the plate and the pin together. As illustrated, the apparatus may also comprise an intermediate sleeve 6. The latter, although not being essential to the invention, is advantageous in that it provides better dimensional control of the hole receiving the blocking sleeve, than if the blocking sleeve 10 were be directly screwed into the hole made by the practitioner in the plate 2.

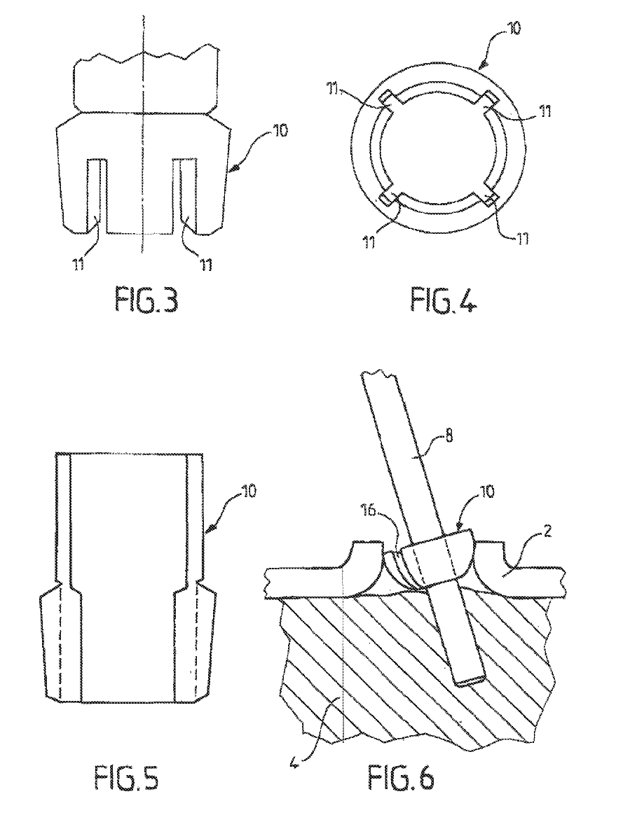

According to the invention, the blocking sleeve comprises at least one slit in its lower portion. As an example, the blocking sleeve may comprise 4 slits as illustrated in the side view of FIG. 3 (only two slits are visible) or in the bottom view of FIG. 4. In certain embodiments, the blocking sleeve comprises at least two slits in its lower portion. In one embodiment, the blocking sleeve comprises from 2 to 6 such slits. Preferably, the lower portion comprises one or more pairs of diametrically opposed slits, more preferably one or two such pairs of slits. The slits divide the lower portion of the blocking sleeve into two or more blades.

Moreover, the intermediate sleeve 6 and the blocking sleeve 10 cooperate by threaded engagement when the blocking sleeve is screwed into the hole defined by the intermediate sleeve.

From a practical point of view, it is possible to proceed in the following way: (a) the blocking sleeve 10 is placed by partly screwing it on the intermediate sleeve 6, the screwing-in being sufficiently slight in order not to interfere with the passage of the pin into the axial orifice of the blocking sleeve 10, (b) the pin 8 is screwed into the bone portion, and (c) the screwing-in of the blocking sleeve 10 is completed. It would also be possible to first screw the pin 8, and then from the top of the pin 8 have the blocking sleeve 10 slide along this pin and screw the blocking sleeve 10 on the intermediate sleeve 6.

In order to facilitate this screwing, the blocking sleeve 10 is provided in its upper portion with a gripping means 12 capable of being set into rotation by means of a screwdriver. This upper portion forms a tool but is not part of the surgical apparatus for osteosynthesis after it has been placed. After the sleeve has been screwed in, its upper portion is removed by shearing at a circular area with less resistance 14.

In the embodiment illustrated in FIG. 2, each of the two sleeves includes a threading. It is understood that this is not mandatory. It is sufficient that one of the sleeves has a thread and that the other sleeve is in a softer material so that the thread meshes into this other sleeve.

In the technical field of the invention, it is for example possible to use non-work-hardened 316L steel which has a tensile strength of the order of 45 kg/mm², for one of the sleeves and work-hardened 316L steel, which has a tensile strength of the order of 145 kg/mm² for the other sleeve. The same steel may also be used for both sleeves and one of them may be hardened at least in its portion intended to cooperate with the other sleeve, by a carbon treatment.

The thickness of the upper portion of the blocking sleeve is selected to suit the material of which the sleeve is made. In an embodiment, the blocking sleeve is made of 316L surgical steel and the upper portion has a wall thickness of about 0.05 mm to about 1.5 mm, more preferably between about 0.1 mm and about 0.6 mm. In one embodiment, the blocking sleeve is made of 316L surgical steel and the upper portion has a wall thickness of about 1 mm.

In an embodiment, the blocking sleeve is made of titanium, titanium alloy, or steel, preferably surgical steel, such as 316L surgical steel. In an embodiment, the blocking sleeve is made of a biodegradable material, such as polylactide (PLA) or another biodegradable polymer.

Preferably, the pin is harder than the lower portion of the blocking sleeve. For example, the pin can be made of a material that is harder than the material which makes up the blocking sleeve.

The plates, blocking sleeves and pins used in the devices of the invention can be provided in a range of sizes. For example, the plates can have a range of thicknesses such as is typical for osteosynthesis plates. For example, in certain embodiments the plates are from about 1 mm to about 2 min thick. Moreover, the plates can have a range of lengths. In certain embodiments, the plates range from about 5 cm to about 15 cm in length and from about 2.5 cm to about 8 cm in width. In certain embodiments, the width of the plate varies along its length, in certain embodiments having a width from about 1 cm to about 2.5 cm near a hole and from about 1 cm to about 2 cm between two holes. The pins can have diameters which are typical for pins used in osteosynthesis devices. In certain embodiments, the pin has a diameter from about 1 mm to about 3 mm, preferably a diameter of from about 1 mm to about 4 mm. In certain embodiments, the blocking sleeve has a diameter ranging from about 3 mm to about 4 mm. In certain embodiments, the blocking sleeve has a diameter of about 0.5 cm. Those of skill in the art can readily select the appropriate plate, blocking sleeve, intermediate sleeve and pin size for the bone parts to be joined.

The osteosynthesis device of the invention preferably comprises a plate with at least two holes configured to join two bone parts, two blocking sleeves as described herein and two pins. However, in certain embodiments, the osteosynthesis device comprises a plate comprising a first means of attachment to a bone part comprising at least one hole, at least one blocking sleeve as described herein and at least one pin. In this embodiment, the device further comprises a second means of attachment to a bone part which is different from the first means. In one embodiment, the second means does not include the blocking sleeve of the invention. The second means can be any means known in the art for attaching a fixing plate to a bone part, for example, a prior art blocking ring and pin, a staple, or a pin which is directly screwed into the bone through the plate.

In preferred embodiments, the pin is a headless pin. Following blocking of the pin by the blocking sleeve and shearing off of the upper portion of the blocking sleeve, the pin is advantageously sheared at the level of the intermediate sleeve, or in the absence of an intermediate sleeve, at the level of the blocking sleeve. Any rough edges formed by shearing of the blocking sleeve and/or the pin can be smoothed by the practitioner, for example, by tapping with a suitable instrument. The practitioner thus cuts the pin to the required length upon installation of the device. Thus, the invention eliminates the need for a stock of pins of varied lengths. In certain embodiments, the blocking sleeve, the pin and, if present, the intermediate sleeve form a pin head.

In embodiments that include an intermediate sleeve, the upper portion of the intermediate sleeve, that is, the portion of the intermediate sleeve that is above the plane of the fixing plate following insertion of the intermediate sleeve into the hole in the fixing plate, is adapted to engage a screwdriver, wrench or other tool suitable for removal of the pin by the practitioner. Preferably, this portion of the blocking sleeve comprises an external driving feature. The external driving feature can be a polygonal casing, such as a square or hexagonal casing, for engaging a tool, such as a screwdriver or wrench. The external driving feature can also comprise two or more notches or slots in the periphery of the intermediate sleeve. In one embodiment, the intermediate sleeve comprises three notches separated by 120°. In another embodiment, the external driving feature comprises more than three notches, for example, four, five or six notches. The notches are preferably evenly spaced; that is, for n notches, adjacent notches are preferably separated by 360/n degrees.

In embodiments of the invention that do not include an intermediate sleeve, the blocking sleeve preferably comprises, at the upper end of the lower portion, an external driving feature as described above.

Advantageously, the blocking sleeve 10 has starting from its lower face (the one facing the bone portion) and upwards an external casing which increases in size. In other words, the diameter D2 of the blocking sleeve in its lower portion is smaller than the diameter D3 in its upper portion. For example, the casing of the sleeve may be conical or hemispherical.

Thus, the blocking sleeve 10 may be engaged by sliding on the pin 8 without any friction or with low friction, into the hole of the plate and it adheres more and more firmly to the intermediate sleeve 6 as it is gradually screwed in. The radial stresses then exerted on the blocking sleeve tend to close the slits of the latter which reduces its diameter at the slits and effectively locks the blocking sleeve on the pin 8.

The variable casing of the blocking sleeve 10 may either be obtained by using a sleeve with a variable diameter and a constant thread, as illustrated in FIG. 2, or by using a sleeve with a constant diameter but with a gradual thread, i.e. the threading of which is radially larger in the upper portion of the sleeve than in the lower portion of the latter, as illustrated in FIG. 5.

Although a blocking sleeve with a variable casing has been described, it is clear that the casing of the blocking sleeve may be constant and that it is the inner casing of the intermediate sleeve which is variable or the casing of the hole made in the plate when the apparatus is without any intermediate sleeve, the latter as indicated above not being an essential component of the apparatus of the invention.

In an embodiment, the invention provides a method of attaching a fixing plate (2) to a bone part (4) in a subject in need of osteosynthesis. The method comprises the steps of:

(a) providing:

(i) a fixing plate (2) comprising at least one hole;

(ii) a threaded fixing pin (8) designed to be screwed into the bone part; and

(iii) a blocking sleeve (10) intended to be screwed into the at least one hole in the plate, said blocking sleeve including an axial orifice intended to allow the passage of a pin to be screwed into the bone part (4), characterized in that, a lower portion of the blocking sleeve includes at least two slits (11), and said blocking sleeve and the associated hole cooperate by threaded engagement in order to block the blocking sleeve in the hole when the sleeve is screwed into the hole; said blocking sleeve further comprising in its upper portion a gripping means (12) and a circular area with less resistance (14) at the lower end of the upper portion;

(b) positioning the fixing plate (2) over the bone part (4) such that the at least one hole of the plate (2) overlays the bone part (4);
(c) drilling a hole in the bone part (4), wherein the hole in the bone part (4) is aligned with the hole in the fixing plate (2) of step (b);
(d) partially screwing the blocking sleeve (10) into the hole of the fixing plate (2);
(e) inserting the fixing pin (8) into the axial orifice of the blocking sleeve and screwing the fixing pin (8) into the hole in the bone part (4) to a depth sufficient to secure the pin in the bone part (4);
(f) screwing the blocking sleeve (10) into the hole of the fixing plate (2) until the upper portion of the blocking sleeve is sheared off at the area of less resistance (14); and
(g) shearing the fixing pin (8) at the level of the blocking sleeve;
thereby affixing the fixing plate (12) to the bone part (14).

In another embodiment, the method of attaching a fixing plate (2) to a bone part (4) in a subject in need of osteosynthesis comprises the steps of:

(a) providing:

(i) a fixing plate (2) comprising at least one hole 0;

(ii) a threaded fixing pin (8) designed to be screwed into the bone part; and

(iii) a blocking sleeve (10) intended to be screwed into the at least one hole in the plate, said blocking sleeve including an axial orifice intended to allow the passage of a pin to be screwed into the bone part (4), characterized in that, a lower portion of the blocking sleeve includes at least two slits (11, 16), and said blocking sleeve and the associated hole cooperate by threaded engagement in order to block the blocking sleeve in the hole when the sleeve is screwed into the hole; said blocking sleeve further comprising in its upper portion a gripping means (12) and a circular area with less resistance (14) at the lower end of the upper portion;

(b) positioning the fixing plate (2) over the bone part (4) such that the at least one hole of the plate (2) overlays the bone part (4);
(c) drilling a hole in the bone part (4), wherein the hole in the bone part (4) is aligned with the hole in the fixing plate (2) of step (b);
(d) inserting the fixing pin (8) through the hole in the fixing plate and screwing the pin into the hole in the bone part (4) to a depth sufficient to secure the pin in the bone part (4);
(e) sliding the blocking sleeve along the fixing pin and into the hole in the fixing plate;
(f) screwing the blocking sleeve (10) into the hole of the fixing plate (2) until the upper portion of the blocking sleeve is sheared off at the area of less resistance (14); and
(g) shearing the fixing pin (8) at the level of the blocking sleeve;
thereby affixing the fixing plate (2) to the bone part (4).

In certain embodiments, the hole in the fixing plate is modified by insertion of an intermediate sleeve before introduction of the fixing pin or the blocking sleeve, In these embodiments, step (a) further comprises providing an intermediate sleeve which is intended to cooperate by threaded engagement with the hole in the fixing plate and with the blocking sleeve. The intermediate sleeve can be provided pre-inserted into the hole, or the practitioner can insert the intermediate sleeve into the hole, for example, by screwing the intermediate sleeve into the hole. In steps (e) and (f) of these embodiments, the blocking sleeve is positioned and screwed into the hole in the plate defined by the intermediate sleeve.

In an embodiment, the process comprises the steps of identifying first and second bone parts in the subject in need of osteosynthesis. In this embodiment, the fixing plate comprises at least two holes, each hole being configured for attachment to one of the bone parts. In this embodiment, steps (a)-(g) are conducted for attachment of the plate to each of the bone parts, thereby fixing and joining the ends of the bone parts. The plate can be affixed to the two bone parts serially or in parallel. In certain embodiments, the plate comprises more than two holes, and the plate is attached to at least one of the bone parts by installation of two or more blocking sleeves and pins according to steps (a)-(g).

One advantage of the present invention is that it allows micromobility of the pin/plate locking In embodiments in which the pin is harder than the lower portion of the blocking sleeve, the blades formed by the slits in the blocking sleeve are strain-hardened by patient motion and collapse slightly. The resulting micromobility enhances bone consolidation. The micromobility also reduces stress on the pins by allowing the transfer of stress from one pin to an adjacent pin and avoiding torsion of the first pin, which is the main cause of pin breakage.

In FIG. 6 another embodiment of an apparatus according to the invention is illustrated, comprising a plate 2, a pin 8 screwed into a bone portion 4, and a blocking sleeve 10 including a slit 16 which, in the illustrated example, extends over the whole height of this sleeve. The hemispherical shape of the blocking sleeve is particularly of interest in that it allows effective blocking even when the pin is not perpendicular to the plane of the plate.

While this invention has been particularly shown and described with references to preferred embodiments thereof, it will be understood by those skilled in the art that various changes in form and details may be made therein without departing from the scope of the invention encompassed by the appended claims.

1. A surgical apparatus for osteosynthesis comprising:
(a) a plate provided with at least one hole intended to join two bone portions;
(b) At least one headless pin; and
(c) At least one substantially axisymmetric blocking sleeve having an upper portion,
i. a lower portion which is intended to be screwed into one of the holes of the plate and includes at least two slits;
ii. a region between said upper and lower portions having decreased resistance compared to said upper and lower portions;
iii. an axial orifice intended to allow the passage of a pin to be screwed into one of the bone portions, characterized in that, for at least one blocking sleeve: the lower portion of the blocking sleeve includes slits (11) along at least four of its radii, and said blocking sleeve and the associated hole cooperate by threaded engagement in order to block the blocking sleeve in the hole when the sleeve is screwed into the hole.

2. A surgical apparatus for osteosynthesis comprising:
(a) a plate provided with at least one hole intended to join two bone portions;
(b) at least one headless pin;
(c) an intermediate sleeve; and
(d) at least one substantially axisymmetric blocking sleeve having,
i. an upper portion;
ii. a lower portion which is intended to be screwed into the intermediate sleeve and includes slits along at least four of its radii;
iii. a region between said upper and lower portions having decreased resistance compared to said upper and lower portions; and
iv. an axial orifice intended to allow the passage of a pin to be screwed into one of the bone portions, characterized in that, for at least one blocking sleeve: the lower portion of the blocking sleeve includes slits (11) along at least four of its radii, and said blocking sleeve and the intermediate sleeve cooperate by threaded engagement in order to block the blocking sleeve in the hole when the sleeve is screwed into the hole.

3. The surgical apparatus of claim 1, wherein the slits of said lower portion are diametrically opposed.

4. The apparatus according to claim 1, wherein the lower portion has a smaller casing than the upper portion.

5. The apparatus according to claim 1, wherein the blocking sleeve has a conical casing.

6. The apparatus according to claim 1, characterized in that said blocking sleeve has a cylindrical casing and the hole has a beveled casing.

7. The apparatus according to claim 1, characterized in that said blocking sleeve and the edges of the holes of the plate have a different hardness.

8. The apparatus according to claim 7, characterized in that one of the parts among said blocking sleeve and an edge of the hole of the plate intended to receive the sleeve is treated in order to increase its hardness.

9. The apparatus according to claim 1, characterized in that the inner casing of the blocking sleeve and the outer casing of the pin are selected so that the sleeve initially engages with the pin without any friction.

10. The apparatus of claim 1, wherein the blocking sleeve is slidable along the length of the pin.

11. A method of attaching a fixing plate (2) to a bone part (4) in a subject in need of osteosynthesis and the method comprises the steps of:
(A) providing:
(i) a fixing plate (2) comprising at least one hole;
(ii) a threaded fixing pin (8) designed to be screwed into the bone part; and
(iii) a blocking sleeve (10) intended to be screwed into the at least one hole in the plate, said blocking sleeve including an axial orifice intended to allow the passage of a pin to be screwed into the bone part (4), characterized in that, a lower portion of the blocking sleeve includes at least two slits (11, 16), and said blocking sleeve and the associated hole cooperate by threaded engagement in order to block the blocking sleeve in the hole when the sleeve is screwed into the hole; said blocking sleeve further comprising in its upper portion a gripping means (12) and a circular area with less resistance (14) at the lower end of the upper portion;
(B) positioning the fixing plate (2) over the bone part (4) such that the at least one hole of the plate (2) overlays the bone part (4);
(C) drilling a hole in the bone part (4), wherein the hole in the bone part (4) is aligned with the hole in the fixing plate (2) of step (B);
(D) (i) (a) partially screwing the blocking sleeve (10) into the at least one hole of the fixing plate (2) and (b) inserting the fixing pin (8) into the axial orifice of the blocking sleeve and screwing the fixing pin (8) into the hole in the bone part (4) to a depth sufficient to secure the pin in the bone part (4); or
(ii) (a) inserting the fixing pin (8) through the hole in the fixing plate and screwing the pin into the hole in the bone part (4) to a depth sufficient to secure the pin in the bone part (4) and (b) sliding the blocking sleeve along the fixing pin and into the hole in the fixing plate;
(E) screwing the blocking sleeve (10) into the hole of the fixing plate (2) until the upper portion of the blocking sleeve is sheared off at the area of less resistance (14); and
(F) shearing the fixing pin (8) at the level of the blocking sleeve;
thereby affixing the fixing plate (12) to the bone part (14).

12. The method of claim 11, wherein step (a) further comprises providing an intermediate sleeve which is intended to cooperate by threaded engagement with the hole in the fixing plate and with the blocking sleeve.

13. The method of claim 12 further comprising the step of inserting the intermediate sleeve into the hole in the fixing plate prior to step (d).

14. The surgical apparatus of claim 2, wherein the slits of said lower portion are diametrically opposed.

15. The apparatus according to claim 2, wherein the lower portion has a smaller casing than the upper portion.

16. The apparatus according to claim 2, wherein the blocking sleeve has a conical casing.

17. The apparatus according to claim 2, characterized in that said blocking sleeve has a cylindrical casing and the hole has a beveled casing.

18. The apparatus according to claim 2, characterized in that the inner casing of the blocking sleeve and the outer casing of the pin are selected so that the sleeve initially engages with the pin without any friction.

19. The apparatus of claim 2, wherein the blocking sleeve is slidable along the length of the pin.
